(19) United States
(12) Patent Application Publication
(10) Pub. No.: US 2014/0031898 A1
(43) Pub. Date: Jan. 30, 2014

(54) Shabbat-Compatible Auditory Prosthesis Systems and Methods

(71) Applicants: Leonid M. Litvak, Los Angeles, CA (US); Lakshmi N. Mishra, Valencia, CA (US); Reuven Knopf, Beit Shemesh (IL)

(72) Inventors: Leonid M. Litvak, Los Angeles, CA (US); Lakshmi N. Mishra, Valencia, CA (US); Reuven Knopf, Beit Shemesh (IL)

(21) Appl. No.: 13/430,449

(22) Filed: Mar. 26, 2012

Related U.S. Application Data
(60) Provisional application No. 61/467,978, filed on Mar. 26, 2011.

(51) Int. Cl. A61F 11/04; A61N 1/36

(52) U.S. Cl. 607/57

(57) ABSTRACT

An exemplary method includes a Shabbat-compatible auditory prosthesis system (a) detecting a transition of a clock signal from being in an on state to being in an off state, (b) directing, in response the transition of the clock signal to being in the off state, a sound processor to turn off and enter an extended off state during which the sound processor remains off for a first predetermined amount of time, and (c) directing, in response to an elapsing of the first predetermined amount of time, the sound processer to turn on and enter a search state for up to a second predetermined amount of time during which the sound processor searches for an implanted auditory prosthesis. Corresponding methods and systems are also disclosed.

### RELATED APPLICATIONS

The present application claims priority under 35 U.S.C. §119(e) to U.S. Provisional Patent Application No. 61/467,978 by Leonid M. Litvak et al., filed on Mar. 26, 2011, and entitled “Shabbat-Compatible Auditory Prosthesis Systems and Methods,” the contents of which are hereby incorporated by reference in their entirety.

### BACKGROUND INFORMATION

Shabbat is the seventh day of the Jewish week and is considered a day of rest in Judaism. Many Jews observe Shabbat by refraining from performing certain activities, such as turning electric devices on or off.

According to some Rabbinic authorities, the use of a conventional auditory prosthesis system (e.g., a cochlear implant system) is not compatible with observance of Shabbat. This is because the implanted auditory prosthesis (e.g., cochlear implant) is turned off when the external speech processor is removed (e.g., at night before going to bed) and turned on when the external processor is put on (e.g., in the morning after waking up).

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary auditory prosthesis system according to principles described herein.

FIG. 2 illustrates a schematic structure of the human cochlea.

FIG. 3 illustrates exemplary components of a Shabbat-compatible cochlear implant system according to principles described herein.

FIG. 4 shows a state diagram that illustrates an exemplary operation of the Shabbat-compatible auditory prosthesis system of FIG. 3 according to principles described herein.

FIGS. 5-6 illustrate exemplary methods of facilitating Shabbat compliance for an auditory prosthesis system user according to principles described herein.

### DETAILED DESCRIPTION

Shabbat-compatible auditory prosthesis systems and methods are described herein. As will be described below, the systems and methods described herein may enable a user to observe Shabbat and other Jewish holidays with similar restrictions while still being able to enjoy the hearing benefits that auditory prosthesis systems provide.

In some examples, a Shabbat-compatible auditory prosthesis system may include an implanted auditory prosthesis and an externally located speech processor configured to control and provide power to the auditory prosthesis. The Shabbat-compatible auditory prosthesis system may further include or implement a clock that may be programmed by a user to have any number of “off” states or periods of arbitrary duration in a twenty-four hour period. For example, a user may program the clock to be in an off state while the user is typically in bed and/or sleeping (e.g., 9:00 pm to 7:00 am). In response to the clock entering the off state, the speech processor is automatically turned off. This, in turn, causes the implanted auditory prosthesis to turn off. With the speech processor and implanted auditory prosthesis already turned off, the user may remove the speech processor without performing an action on the Shabbat that directly turns either device off. This lack of direct human intervention may make the use of an auditory prosthesis system on the Shabbat acceptable to some Jews.

In some examples, a user may desire to use an auditory prosthesis system while the clock is in the off state. For example, the user may wake up earlier than normal (e.g., before 7:00 am) and desire to use the auditory prosthesis system before the clock switches back to an on state (which causes the speech processor to turn back on). To accommodate this desire, as will be described in more detail below, the Shabbat-compatible auditory prosthesis system described herein may be further configured to direct the speech processor to periodically turn on for relatively short periods of time and attempt to locate the implanted auditory prosthesis while the clock is in the off state. If the implanted auditory prosthesis is found (signifying, e.g., that the user has put the speech processor back on his or her ear), the speech processor automatically connects to (and turns on) the implanted auditory prosthesis. In this manner, the user may put the speech processor on his or her ear while the speech processor is turned off and wait (e.g., up to a few minutes) for the speech processor and implanted auditory prosthesis to turn on automatically. Because the placing of the speech processor on the ear does not directly turn on either the speech processor or the implanted auditory prosthesis, Shabbat compliance may be maintained.

As used herein, “removing” or “taking off” a sound processor refers to an action performed by a person that physically removes the sound processor from being located on or behind the ear of an auditory prosthesis user or that disconnects the sound processor from a headpiece configured to facilitate communication between the sound processor and an implanted auditory prosthesis. For example, a user may remove or take off a sound processor before going to bed. Likewise, “putting on” a sound processor refers to an action performed by a person that places the sound processor on or behind the ear or that connects the sound processor to the headpiece. For example, a user may put on a sound processor after getting out of bed after sleeping.

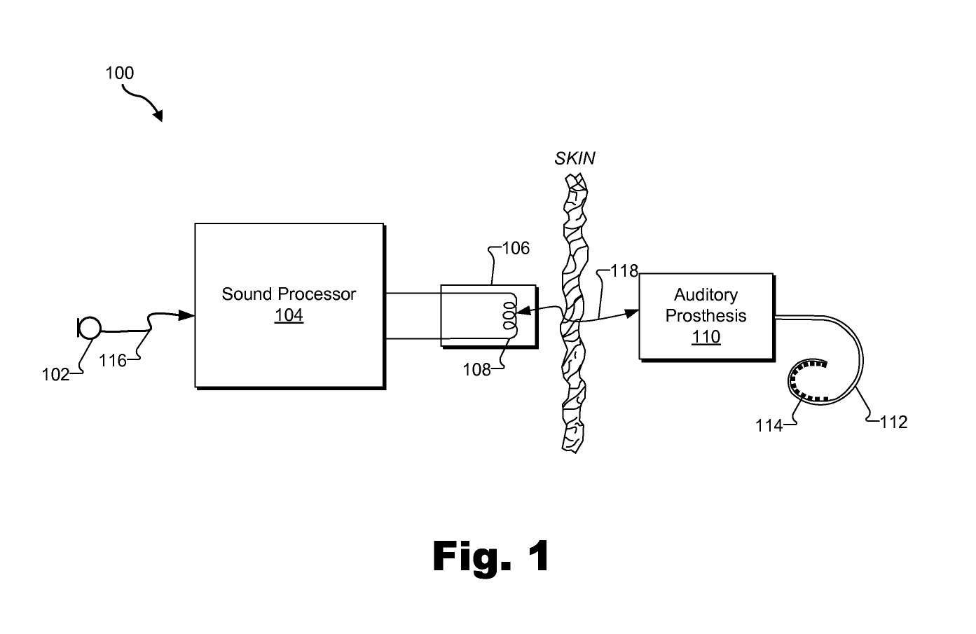

FIG. 1 illustrates an exemplary auditory prosthesis system 100. Auditory prosthesis system 100 may include a microphone 102, a sound processor 104, a headpiece 106 having a coil 108 disposed therein, an auditory prosthesis 110, and a lead 112 with a plurality of electrodes 114 disposed thereon. Additional or alternative components may be included within auditory prosthesis system 100 as may serve a particular implementation.

As shown in FIG. 1, microphone 102, sound processor 104, and headpiece 106 may be located external to an auditory prosthesis patient. In some alternative examples, microphone 102 and/or sound processor 104 may be implanted within the patient. In such configurations, the need for headpiece 106 may be obviated.

Microphone 102 may detect an audio signal and convert the detected signal to a corresponding electrical signal. The electrical signal may be sent from microphone 102 to sound processor 104 via a communication link 116, which may include a telemetry link, a wire, and/or any other suitable communication link.

Sound processor 104 is configured to direct auditory prosthesis 110 to generate and apply electrical stimulation (also referred to herein as “stimulation current”) to one or more stimulation sites associated with an auditory pathway (e.g., the auditory nerve) of the patient. Exemplary stimulation sites include, but are not limited to, one or more locations within the cochlea, the cochlear nucleus, the inferior colliculus, and/or any other nuclei in the auditory pathway. To this end, sound processor 104 may process the audio signal detected by microphone 102 in accordance with a selected sound processing strategy to generate appropriate stimulation parameters for controlling auditory prosthesis 110. Sound processor 104 may include or be implemented by a behind-the-ear (“BTE”) unit, a portable speech processor (“PSP”), and/or any other sound processing unit as may serve a particular implementation.

Sound processor 104 may be configured to transcutaneously transmit one or more control parameters and/or one or more power signals to auditory prosthesis 110 with coil 108 by way of a communication link 118. These control parameters may be configured to specify one or more stimulation parameters, operating parameters, and/or any other parameter by which auditory prosthesis 110 is to operate as may serve a particular implementation. Exemplary control parameters include, but are not limited to, stimulation current levels, volume control parameters, program selection parameters, operational state parameters (e.g., parameters that turn a sound processor and/or an auditory prosthesis on or off), audio input source selection parameters, fitting parameters, noise reduction parameters, microphone sensitivity parameters, microphone direction parameters, pitch parameters, timbre parameters, sound quality parameters, most comfortable current levels (“M levels”), threshold current levels, channel acoustic gain parameters, front and backend dynamic range parameters, current steering parameters, pulse rate values, pulse width values, frequency parameters, amplitude parameters, waveform parameters, electrode polarity parameters (i.e., anode-cathode assignment), location parameters (i.e., which electrode pair or electrode group receives the stimulation current), stimulation type parameters (i.e., monopolar, bipolar, or tripolar stimulation), burst pattern parameters (e.g., burst on time and burst off time), duty cycle parameters, spectral tilt parameters, filter parameters, and dynamic compression parameters. Sound processor 104 may also be configured to operate in accordance with one or more of the control parameters.

As shown in FIG. 1, coil 108 may be housed within headpiece 106, which may be affixed to a patient's head and positioned such that coil 108 is communicatively coupled to a corresponding coil included within auditory prosthesis 110. In this manner, control parameters and power signals may be wirelessly transmitted between sound processor 104 and auditory prosthesis 110 via communication link 118. It will be understood that data communication link 118 may include a bi-directional communication link and/or one or more dedicated uni-directional communication links. In some alternative embodiments, sound processor 104 and auditory prosthesis 110 may be directly connected with one or more wires or the like.

Auditory prosthesis 110 may include any type of implantable stimulator that may be used in association with the systems and methods described herein. For example, auditory prosthesis 110 may include an implantable cochlear stimulator. In some alternative implementations, auditory prosthesis 110 may include a brainstem implant and/or any other type of auditory prosthesis that may be implanted within a patient and configured to apply stimulation to one or more stimulation sites located along an auditory pathway of a patient.

In some examples, auditory prosthesis 110 may be configured to generate electrical stimulation representative of an audio signal detected by microphone 102 in accordance with one or more stimulation parameters transmitted thereto by sound processor 104. Auditory prosthesis 110 may be further configured to apply the electrical stimulation to one or more stimulation sites within the patient via one or more electrodes 114 disposed along lead 112. In some examples, auditory prosthesis 110 may include a plurality of independent current sources each associated with a channel defined by one or more of electrodes 114. In this manner, different stimulation current levels may be applied to multiple stimulation sites simultaneously by way of multiple electrodes 114. In such examples, auditory prosthesis system 100 may be referred to as a “multi-channel auditory prosthesis system.”

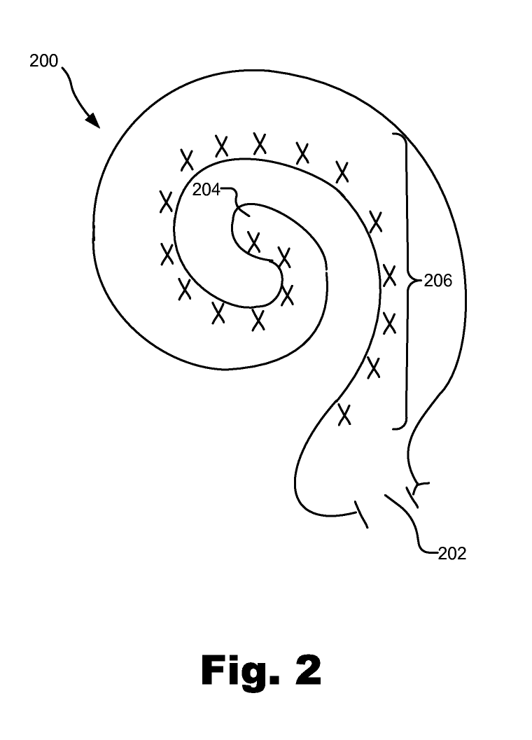

To facilitate application of the electrical stimulation generated by auditory prosthesis 110, lead 112 may be inserted within a duct of the cochlea such that electrodes 114 are in communication with one or more stimulation sites within the cochlea. FIG. 2 illustrates a schematic structure of the human cochlea 200 into which lead 112 may be inserted. As shown in FIG. 2, the cochlea 200 is in the shape of a spiral beginning at a base 202 and ending at an apex 204. Within the cochlea 200 resides auditory nerve tissue 206, which is denoted by Xs in FIG. 2. The auditory nerve tissue 206 is organized within the cochlea 200 in a tonotopic manner. Low frequencies are encoded at the apex 204 of the cochlea 200 while high frequencies are encoded at the base 202. Hence, each location along the length of the cochlea 200 corresponds to a different perceived frequency. Auditory prosthesis system 100 may therefore be configured to apply electrical stimulation to different locations within the cochlea 200 (e.g., different locations along the auditory nerve tissue 206) to provide a sensation of hearing.

Alternatively, lead 112 may be implanted within a patient such that electrodes 114 are in communication with one or more stimulation sites otherwise located along the auditory pathway. As used herein, the term “in communication with” refers to electrodes 114 being adjacent to, in the general vicinity of, in close proximity to, directly next to, or directly on the stimulation site. Any number of electrodes 114 (e.g., sixteen) may be disposed on lead 112 as may serve a particular implementation.

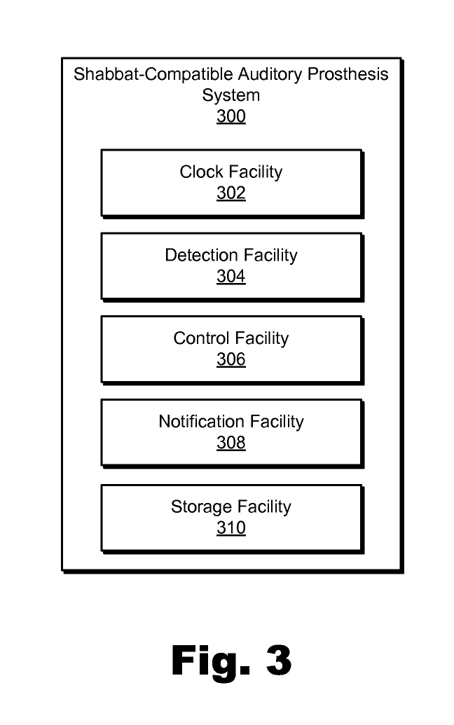

FIG. 3 illustrates exemplary components of a Shabbat-compatible cochlear implant system 300 (or simply “system 300”). As shown in FIG. 3, system 300 may include a clock facility 302, a detection facility 304, a control facility 306, a notification facility 308, and a storage facility 310, any or all of which may be in communication with one another using any suitable communication technologies. Each of these facilities 302-310 may include any combination of hardware, software, and/or firmware as may serve a particular implementation. For example, one or more of facilities 302-310 may be implemented by sound processor 104, one or more accessories coupled to sound processor 104, and/or any other computing device or processor as may serve a particular implementation. Facilities 302-310 will now be described in more detail.

Clock facility 302 may be configured to generate a clock signal having any number of on and off states of user-programmable duration in a twenty-four hour period. To this end, clock facility 302 may accept user input and adjust the number of on and off states and/or a duration of the on and off states in accordance with the user input.

For example, a user (e.g., a user of system 300 and/or an audiologist) may program the clock signal to be in two off states during a particular twenty-four hour period (e.g., between 9:00 pm and 7:00 am and between 1:00 pm and 3:00 pm). As will be described below, control facility 306 may automatically turn speech processor 104 off in response to the clock signal entering each off state. Likewise, control facility 306 may automatically turn speech processor 104 on in response to the clock signal entering an on state.

In some examples, clock facility 302 is implemented by a clock that tracks actual times and dates. Alternatively, clock facility 302 may be implemented by a timer that starts when speech processor 104 is turned on before Shabbat. The timer may be configured to expire shortly before Shabbat commences, thereby causing speech processor 104 to be turned off. After speech processor 104 is turned off, the timer may start again for a predetermined amount of time. Upon expiration of the timer, speech processor 104 may be turned back on.

Detection facility 304 may be configured to detect a transition of the clock signal from being in an on state to being in an off state. Detection facility 304 may be further configured to detect a transition of the clock signal from being in an off state to being in an on state. In response to these transitions, speech processor 104 may be directed to enter various operational states, as will be described below.

Control facility 304 may be configured to direct speech processor 104 to enter various operational states in which speech processor 104 operates normally, is turned off, is turned on for short periods of time, and/or searches for an auditory prosthesis to which it may connect. To illustrate, in response to a transition of the clock signal to being in the off state, control facility 304 may direct sound processor 104 to turn off and enter an extended off state during which sound processor 104 remains off for a predetermined amount of time. Other operational states into which sound processor 104 may enter will be described in more detail below.

Notification facility 308 may be configured to notify a user of system 300 that sound processor 104 has entered and/or is in a particular operational state. In this manner, the user may know when he or she can remove or put on sound processor 104 without violating Shabbat. To this end, notification facility 308 may be implemented by one or more light emitting diodes (“LEDs”), speakers, and/or any other notification means as may serve a particular implementation. Examples of various types of notifications that may be provided by notification facility 308 will be provided below.

Storage facility 310 may be configured to maintain data generated and/or utilized by clock facility 302, detection facility 304, control facility 306, and/or notification facility 308. Storage facility 310 may be configured to maintain additional or alternative data as may serve a particular implementation.

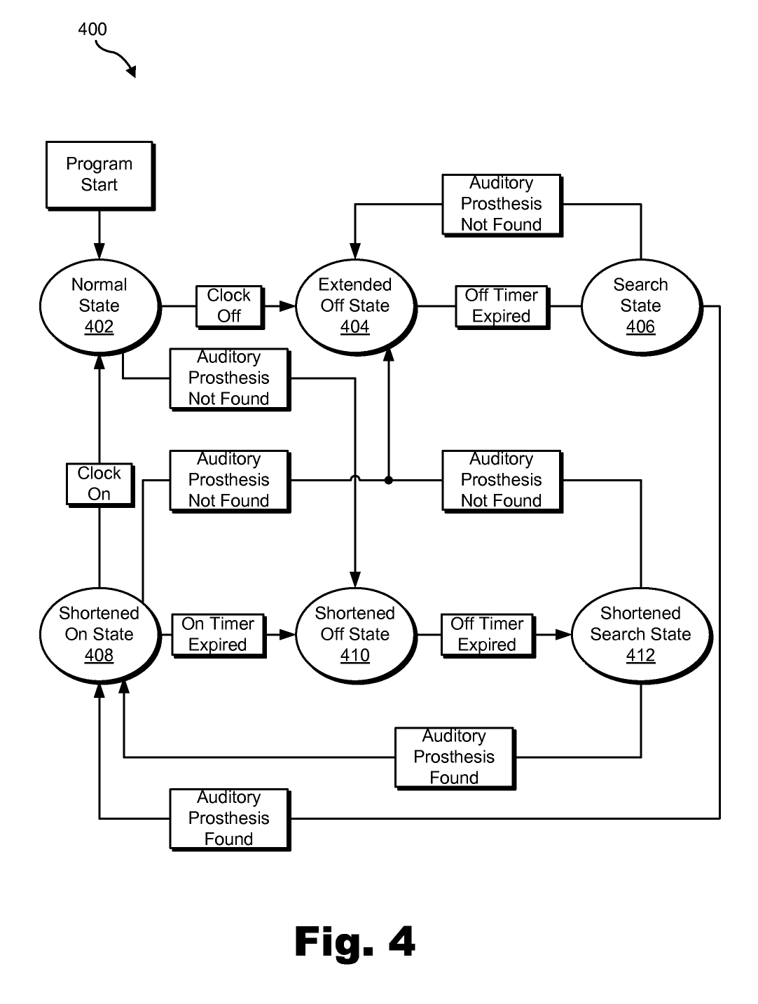

FIG. 4 shows a state diagram 400 that illustrates an exemplary operation of Shabbat-compatible auditory prosthesis system 300. It will be recognized that state diagram 400 is merely illustrative of the many different manners in which Shabbat-compatible auditory prosthesis system 300 may operate. Each of the states shown in state diagram 400 represent different operational states of sound processor 104.

As shown, after starting a program (e.g., by turning on), sound processor 104 may be in a normal state 402 during which sound processor 104 may remain on until the clock signal transitions to being in an off state (“clock off”) or until auditory prosthesis 110 cannot be found (“auditory prosthesis not found”). If auditory prosthesis 110 is not found (which may signify that sound processor 104 has been removed and/or that sound processor 104 has stopped providing power to auditory prosthesis 100 for any other reason), system 300 may direct sound processor 104 to enter a shortened off state 410, which will be described in more detail below.

In response to the transition of the clock signal to being in the off state, system 300 (e.g., control facility 306) may direct sound processor 104 to turn off and enter an extended off state 404 during which sound processor 104 remains off for a predetermined amount of time. The predetermined amount of time may include any amount of time (e.g., three minutes) and, in some examples, may be specified by a user. In some examples, an “off timer” may keep track of the predetermined amount of time.

In some examples, notification facility 108 may notify the user when sound processor 104 enters the extended off state 404. For example, notification facility 108 may direct sound processor 104 to emit an audible signal (e.g., a beep), emit any other type of predetermined electric signal, and/or turn off an LED that is a part of sound processor 104 when sound processor 104 turns off and enters the extended off state 404. In this manner, the user may know that sound processor 104 has entered the extended off state 404 and that it is safe to remove sound processor 104 without violating Shabbat.

As mentioned, in some examples, a user may desire to use an auditory prosthesis system while the clock is in the off state. For example, the user may wake up earlier than normal and desire to use the auditory prosthesis system before the clock signal switches back to an on state. Hence, while sound processor 104 is in extended off state 404, the user may put sound processor 104 on his or her ear and wait for the predetermined amount of time to elapse. As shown in FIG. 4, in response to an elapsing of the predetermined amount of time (“off timer expired”), system 300 may direct sound processor 104 to turn on and enter a search state 406 for up to another predetermined amount of time during which sound processor 104 searches for auditory prosthesis 110. If auditory prosthesis 110 is located, system 300 may direct sound processor 104 to enter a shortened on state 408 during which the user may enjoy the hearing benefits of system 300. Shortened on state 408 will be described in more detail below.

However, if sound processor 104 does not locate auditory prosthesis 110 while in the search state 406 (thus signifying that the user has not placed sound processor 104 on his or her ear), system 100 may direct sound processor 104 to return to the extended off state 404. Sound processor 104 may iteratively alternate from being in extended off state 404 and search state 406 until auditory prosthesis 110 is found (thus signifying that the user has placed sound processor 104 on his or her ear) or until the clock signal returns to the on state (whereupon sound processor 104 would return to normal state 402).

The predetermined amount of time associated with search state 406 may include any amount of time (e.g., a minute). In some examples, notification facility 108 may illuminate an LED that is a part of sound processor 104 while sound processor 104 is in the search state 406 so that the user knows that he or she should not put sound processor 104 on his or her ear (and thereby turn on auditory prosthesis 110, which would be a violation of Shabbat).

As mentioned, if sound processor 104 locates auditory prosthesis 110 while in search state 406, system 300 may direct sound processor 104 to enter the shortened on state 408. While in the shortened on state 408, sound processor 104 remains on for a relatively short amount of time (e.g., fifteen minutes). The amount of time during which sound processor 104 remains on during the shortened on state 408 may be specified by the user and may be tracked by an “on timer.”

In some examples, system 300 may be configured to direct sound processor 104 to periodically exit the shortened on state (e.g., by turning off) and enter a shortened off state 410 during which sound processor remains off for a predetermined amount of time. For example, as shown in FIG. 4, in response to an “on time expired” event, system 300 may direct sound processor 104 to turn off and enter shortened off state 410. In some examples, notification facility 108 may direct sound processor 104 to emit an audible signal (e.g., a beep) prior to sound processor 104 turning off and entering shortened off state 410 so that the user may know that sound processor 104 is about to turn off.

In some examples, the predetermined amount of time associated with the shortened off state 410 is less than the predetermined amount of time associated with the extended off state 410. For example, the predetermined amount of time associated with the shortened off state 410 may be equal to ten seconds while the predetermined amount of time associated with the extended off state 410 may be equal to fifteen minutes, as described previously.

In response to an elapsing of predetermined amount of time associated with the shortened off state 410 (“off timer expired”), system 100 may direct sound processor 104 to turn back on and enter a shortened search state 412 during which sound processor 104 searches for auditory prosthesis 110. In some examples, notification facility 108 may illuminate an LED that is a part of sound processor 104 while sound processor 104 is in shortened search state 412 so that the user knows that he or she should not put sound processor 104 on his or her ear (and thereby turn on auditory prosthesis 110, which would be a violation of Shabbat).

If auditory prosthesis 110 is not located during the shortened search state 412 (which may signify that the user has removed sound processor 104 during the shortened off state 410), system 300 may direct sound processor 104 to return to extended off state 404. However, if auditory prosthesis 110 is found, system 300 may direct sound processor 104 to return to shortened on state 408.

Shortened on state 408, shortened off state 410, and shortened search state 412 may allow a user to use system 300 even while the clock signal is in an off state by periodically giving the user a chance to remove or put on sound processor 104 while sound processor 104 is turned off (i.e., while sound processor 104 is in shortened off state 410). Hence, system 300 may iteratively direct sound processor 104 to be in shortened on state 408, shortened off state 410, and shortened search state 412 until auditory prosthesis 110 is not found (at which point sound processor 104 returns to extended off state 404) or until the clock signal transitions back to an on state (at which point sound processor 104 returns to normal state 402).

As mentioned, some other Jewish holidays have similar restrictions to Shabbat. In some cases, one or more of these holidays may be right before or after Shabbat such that it may be desirable for a user to comply with Shabbat regulations for any number of consecutive days in a row (e.g., three days in a row). Hence, sound processor 104 may be equipped with a battery that is configured to provide enough power to last up to three or more days without having to be recharged.

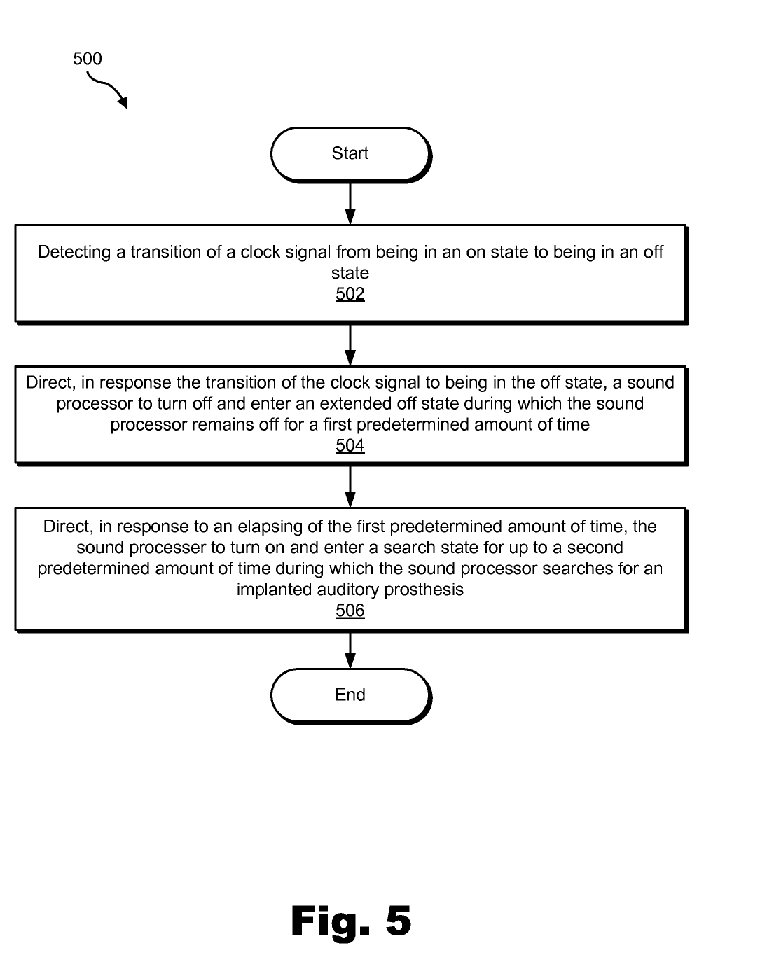

FIG. 5 illustrates an exemplary method 500 of facilitating Shabbat compliance for an auditory prosthesis system user. While FIG. 5 illustrates exemplary steps according to one embodiment, other embodiments may omit, add to, reorder, and/or modify any of the steps shown in FIG. 5. One or more of the steps shown in FIG. 5 may be performed by any component or combination of components of Shabbat-compatible auditory prosthesis system 300 and/or sound processor 104.

In step 502, a transition of a clock signal from being in an on state to being in an off state is detected. Step 502 may be performed in any of the ways described herein.

In step 504, in response the transition of the clock signal to being in the off state, a sound processor is directed to turn off and enter an extended off state during which the sound processor remains off for a first predetermined amount of time. Step 504 may be performed in any of the ways described herein.

In step 506, in response to an elapsing of the first predetermined amount of time, the sound processer is directed to turn on and enter a search state for up to a second predetermined amount of time during which the sound processor searches for an implanted auditory prosthesis. Step 506 may be performed in any of the ways described herein.

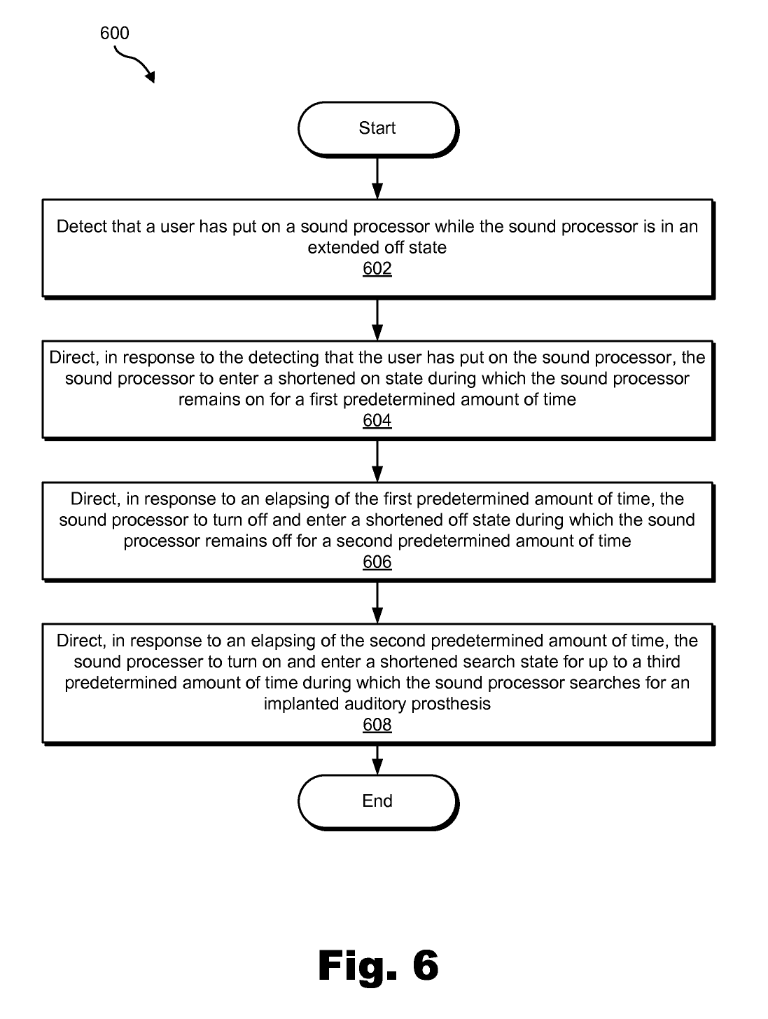

FIG. 6 illustrates another exemplary method 600 of facilitating Shabbat compliance for an auditory prosthesis system user. While FIG. 6 illustrates exemplary steps according to one embodiment, other embodiments may omit, add to, reorder, and/or modify any of the steps shown in FIG. 6. One or more of the steps shown in FIG. 6 may be performed by any component or combination of components of Shabbat-compatible auditory prosthesis system 300.

In step 602, a Shabbat-compatible auditory prosthesis system detects that a user has put on a sound processor while the sound processor is in an extended off state. Step 602 may be performed in any of the ways described herein.

In step 604, the Shabbat-compatible auditory prosthesis system directs, in response to the detecting that the user has put on the sound processor, the sound processor to enter a shortened on state during which the sound processor remains on for a first predetermined amount of time. Step 604 may be performed in any of the ways described herein.

In step 606, the Shabbat-compatible auditory prosthesis system directs, in response to an elapsing of the first predetermined amount of time, the sound processor to turn off and enter a shortened off state during which the sound processor remains off for a second predetermined amount of time. Step 606 may be performed in any of the ways described herein.

In step 608, the Shabbat-compatible auditory prosthesis system directs, in response to an elapsing of the second predetermined amount of time, the sound processer to turn on and enter a shortened search state for up to a third predetermined amount of time during which the sound processor searches for an implanted auditory prosthesis. Step 608 may be performed in any of the ways described herein.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a floppy disk, flexible disk, hard disk, magnetic tape, any other magnetic medium, a CD-ROM, DVD, any other optical medium, a RAM, a PROM, an EPROM, a FLASH-EEPROM, any other memory chip or cartridge, or any other tangible medium from which a computer can read.

As detailed above, system 300 may perform one or more steps of an exemplary method of facilitating Shabbat-compliance by an auditory prosthesis user. In one example, such a method may include (a) detecting, by a Shabbat-compatible auditory prosthesis system, a transition of a clock signal from being in an on state to being in an off state, (b) directing, by the Shabbat-compatible auditory prosthesis system in response the transition of the clock signal to being in the off state, a sound processor to turn off and enter an extended off state during which the sound processor remains off for a first predetermined amount of time, and (c) directing, by the Shabbat-compatible auditory prosthesis system in response to an elapsing of the first predetermined amount of time, the sound processer to turn on and enter a search state for up to a second predetermined amount of time during which the sound processor searches for an implanted auditory prosthesis.

In some examples, the method may further include (d) determining, by the Shabbat-compatible auditory prosthesis system, that the sound processor does not locate the implanted auditory prosthesis during the second predetermined amount of time, and (e) directing, by the Shabbat-compatible auditory prosthesis system in response to the determining, the sound processor to turn off and return to the extended off state. In some examples, the method may further include iteratively repeating, by the Shabbat-compatible auditory prosthesis system, steps (c), (d), and (e) until the sound processor locates the implanted auditory prosthesis or until the clock signal transitions from the off state back to the on state.

Alternatively, the method may further include (d) determining, by the Shabbat-compatible auditory prosthesis system, that the sound processor has located the implanted auditory prosthesis during the second predetermined amount of time, (e) directing, by the Shabbat-compatible auditory prosthesis system in response to the determining, the sound processor to connect to the implanted auditory prosthesis and enter a shortened on state during which the sound processor remains on for a third predetermined amount of time, (f) directing, by the Shabbat-compatible auditory prosthesis system in response to an elapsing of the third predetermined amount of time, the sound processor to turn off and enter a shortened off state during which the sound processor remains off for a fourth predetermined amount of time, and (g) directing, by the Shabbat-compatible auditory prosthesis system in response to an elapsing of the fourth predetermined amount of time, the sound processer to turn on and enter a shortened search state for up to a fifth predetermined amount of time during which the sound processor searches for the implanted auditory prosthesis.

In some examples, the method may further include (h) determining, by the Shabbat-compatible auditory prosthesis system, that the sound processor does not locate the implanted auditory prosthesis during the fifth predetermined amount of time, and (i) directing, by the Shabbat-compatible auditory prosthesis system in response to the determining that the sound processor does not locate the implanted auditory prosthesis during the fifth predetermined amount of time, the sound processor to turn off and return to the extended off state.

Alternatively, the method may further include (h) determining, by the Shabbat-compatible auditory prosthesis system, that the sound processor has located the implanted auditory prosthesis during the fifth predetermined amount of time, and (i) directing, by the Shabbat-compatible auditory prosthesis system in response to the determining that the sound processor has located the implanted auditory prosthesis during the fifth predetermined amount of time, the sound processor to connect to the implanted auditory prosthesis and return to the shortened on state. In some examples, steps (f), (g), (h), and (i) may be iteratively repeated until the sound processor does not locate the implanted auditory prosthesis within the fifth predetermined amount of time associated with the shortened search state.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A method comprising:
(a) detecting, by a Shabbat-compatible auditory prosthesis system, a transition of a clock signal from being in an on state to being in an off state;
(b) directing, by the Shabbat-compatible auditory prosthesis system in response the transition of the clock signal to being in the off state, a sound processor to turn off and enter an extended off state during which the sound processor remains off for a first predetermined amount of time; and
(c) directing, by the Shabbat-compatible auditory prosthesis system in response to an elapsing of the first predetermined amount of time, the sound processer to turn on and enter a search state for up to a second predetermined amount of time during which the sound processor searches for an implanted auditory prosthesis.

2. The method of claim 1, further comprising:
(d) determining, by the Shabbat-compatible auditory prosthesis system, that the sound processor does not locate the implanted auditory prosthesis during the second predetermined amount of time; and
(e) directing, by the Shabbat-compatible auditory prosthesis system in response to the determining, the sound processor to turn off and return to the extended off state.

3. The method of claim 2, further comprising iteratively repeating, by the Shabbat-compatible auditory prosthesis system, steps (c), (d), and (e) until the sound processor locates the implanted auditory prosthesis or until the clock signal transitions from the off state back to the on state.

4. The method of claim 1, further comprising:
(d) determining, by the Shabbat-compatible auditory prosthesis system, that the sound processor has located the implanted auditory prosthesis during the second predetermined amount of time; and
(e) directing, by the Shabbat-compatible auditory prosthesis system in response to the determining, the sound processor to connect to the implanted auditory prosthesis and enter a shortened on state during which the sound processor remains on for a third predetermined amount of time.

5. The method of claim 4, further comprising:
(f) directing, by the Shabbat-compatible auditory prosthesis system in response to an elapsing of the third predetermined amount of time, the sound processor to turn off and enter a shortened off state during which the sound processor remains off for a fourth predetermined amount of time.

6. The method of claim 5, further comprising:
(g) directing, by the Shabbat-compatible auditory prosthesis system in response to an elapsing of the fourth predetermined amount of time, the sound processer to turn on and enter a shortened search state for up to a fifth predetermined amount of time during which the sound processor searches for the implanted auditory prosthesis.

7. The method of claim 6, further comprising:
(h) determining, by the Shabbat-compatible auditory prosthesis system, that the sound processor does not locate the implanted auditory prosthesis during the fifth predetermined amount of time; and
(i) directing, by the Shabbat-compatible auditory prosthesis system in response to the determining that the sound processor does not locate the implanted auditory prosthesis during the fifth predetermined amount of time, the sound processor to turn off and return to the extended off state.

8. The method of claim 6, further comprising:
(h) determining, by the Shabbat-compatible auditory prosthesis system, that the sound processor has located the implanted auditory prosthesis during the fifth predetermined amount of time; and
(i) directing, by the Shabbat-compatible auditory prosthesis system in response to the determining that the sound processor has located the implanted auditory prosthesis during the fifth predetermined amount of time, the sound processor to connect to the implanted auditory prosthesis and return to the shortened on state.

9. The method of claim 8, further comprising iteratively repeating steps (f), (g), (h), and (i) until the sound processor does not locate the implanted auditory prosthesis within the fifth predetermined amount of time associated with the shortened search state.

10. The method of claim 6, further comprising:
(h) illuminating, by the Shabbat-compatible auditory prosthesis system, an LED while the sound processor is in the shortened search state.

11. The method of claim 4, further comprising:
(f) directing, by the Shabbat-compatible auditory prosthesis system, the sound processor to emit an audible signal in association with the sound processor turning off and entering the shortened off state.

12. The method of claim 1, further comprising:
(d) detecting, by a Shabbat-compatible auditory prosthesis system, a transition of the clock signal from being in the off state back to being in the on state;
(e) directing, by the Shabbat-compatible auditory prosthesis system in response to the detecting, the sound processor turn on and enter a normal state during which the sound processor remains on until the clock signal transitions back to being in the off state.

13. The method of claim 1, further comprising:
(d) directing, by the Shabbat-compatible auditory prosthesis system, the sound processor to emit an audible signal in association with the sound processor turning off and entering the extended off state.

14. The method of claim 1, further comprising:
(d) illuminating, by the Shabbat-compatible auditory prosthesis system, an LED while the sound processor is in the search state.

15. The method of claim 1, wherein the clock signal is user-programmable.

16. The method of claim 1, embodied as computer-executable instructions on at least one non-transitory computer-readable medium.

17. A method comprising:
detecting, by a Shabbat-compatible auditory prosthesis system that a user has put on a sound processor while the sound processor is in an extended off state;
directing, by the Shabbat-compatible auditory prosthesis system in response to the detecting that the user has put on the sound processor, the sound processor to enter a shortened on state during which the sound processor remains on for a first predetermined amount of time;
directing, by the Shabbat-compatible auditory prosthesis system in response to an elapsing of the first predetermined amount of time, the sound processor to turn off and enter a shortened off state during which the sound processor remains off for a second predetermined amount of time; and
directing, by the Shabbat-compatible auditory prosthesis system in response to an elapsing of the second predetermined amount of time, the sound processer to turn on and enter a shortened search state for up to a third predetermined amount of time during which the sound processor searches for an implanted auditory prosthesis.

18. The method of claim 17, further comprising:
determining, by the Shabbat-compatible auditory prosthesis system, that the sound processor does not locate the implanted auditory prosthesis during the third predetermined amount of time; and
directing, by the Shabbat-compatible auditory prosthesis system in response to the determining that the sound processor does not locate the implanted auditory prosthesis during the third predetermined amount of time, the sound processor to turn off and return to the extended off state.

19. The method of claim 17, further comprising:
determining, by the Shabbat-compatible auditory prosthesis system, that the sound processor has located the implanted auditory prosthesis during the third predetermined amount of time; and
directing, by the Shabbat-compatible auditory prosthesis system in response to the determining that the sound processor has located the implanted auditory prosthesis during the third predetermined amount of time, the sound processor to connect to the implanted auditory prosthesis and return to the shortened on state.

20. A Shabbat-compatible auditory prosthesis system comprising:
a detection facility configured to detect a transition of a clock signal from being in an on state to being in an off state; and
a control facility communicatively coupled to the detection facility and configured to
direct, in response the transition of the clock signal to being in the off state, a sound processor to turn off and enter an extended off state during which the sound processor remains off for a first predetermined amount of time, and
direct, in response to an elapsing of the first predetermined amount of time, the sound processer to turn on and enter a search state for up to a second predetermined amount of time during which the sound processor searches for an implanted auditory prosthesis.
